(19) United States
(12) Plant Patent Application Publication
(10) Pub. No.: US 2008/0148460 P1
(43) Pub. Date: Jun. 19, 2008

(54) Sanvitalia speciosa plant named 'Sandeal'

(75) Inventor: Theodorus Cornelis Maria van Kleinwee, Hoorn (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/641,406

(22) Filed: Dec. 19, 2006

(51) Int. Cl. A01H 5/00

(52) U.S. Cl. PLT/476

(57) ABSTRACT

A distinct cultivar of Sanvitalia plant named ‘Sandeal’ which is particularly distinguished by its very compact plant habit, ball shape, small flowers, and constant floridity during summer.

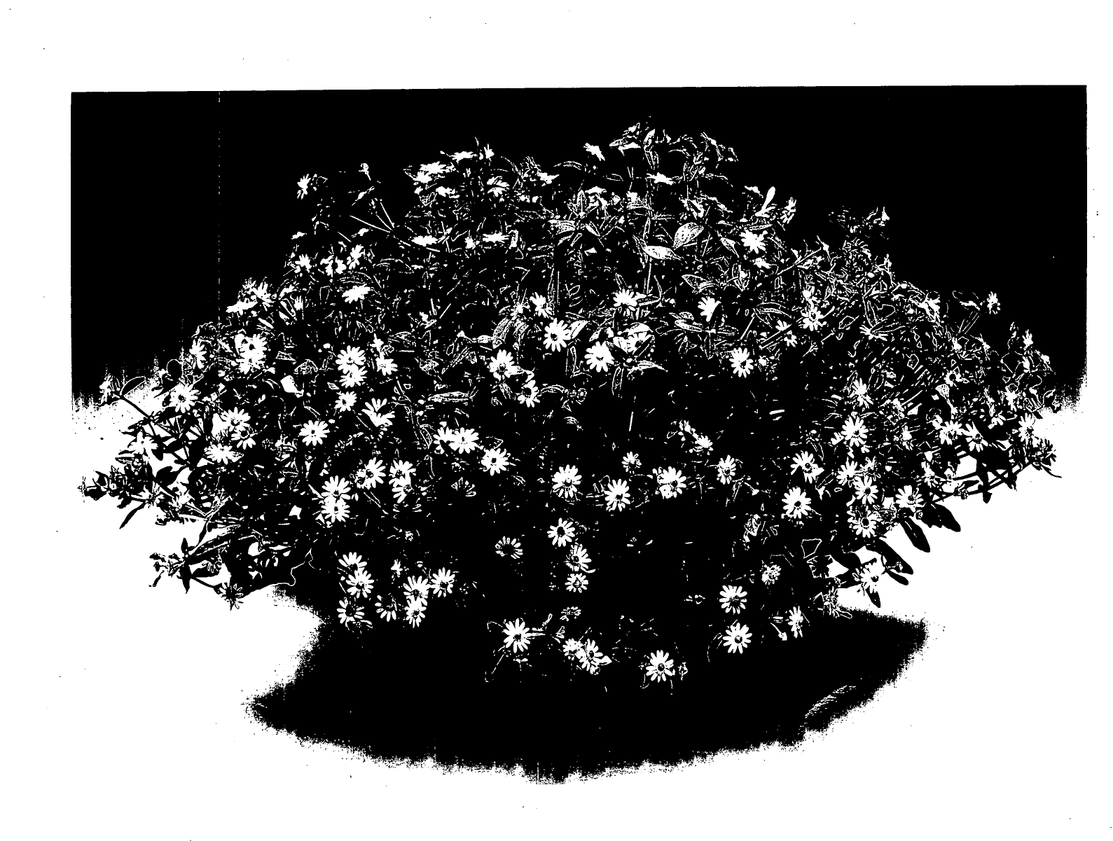

### LATIN NAME OF THE GENUS AND SPECIES OF THE PLANT CLAIMED

Sanvitalia x hybrida

### VARIETAL DENOMINATION

‘Sandeal’

### BACKGROUND OF THE NEW PLANT

The present invention comprises a new distinct cultivar of Sanvitalia, botanically known as Sanvitalia x hybrida, native to South USA and Mexico. The new cultivar is propagated by cuttings resulting from a seedling from female plant Sanvitalia x hybrida identified as number ‘A0178A-1.’ The male parent is unidentified as the seedling was a result of a non-controlled pollination. Seed of the female parent was harvested in 1999 and the new variety ‘Sandeal’ was selected in 2000 as one of the plants of this population. This new plant is an annual. The new cultivar is a product of a planned breeding program conducted by the inventor in Enkhuizen, The Netherlands. The new cultivar ‘Sandeal’ is a compact Sanvitalia hybrid that flowers throughout the summer. ‘Sandeal’ was asexually reproduced by cuttings for 6 years now and trialed under different conditions in Europe on Syngenta trial ground properties. It has been found to retain its distinctiveness characteristics through successive propagation and its novelty is firmly fixed. The new variety reproduces true to type in successive generations of asexual reproduction.

### DESCRIPTION OF THE DRAWING

This new Sanvitalia plant is illustrated by the accompanying photographic drawing which shows blooms, buds and foliage of the plant in full color, the color shown being as true as can be reasonably obtained by conventional photographic procedures.

### DESCRIPTION OF THE NEW CULTIVAR

The following detailed descriptions set forth the distinctive characteristics of this new Sanvitalia plant. The data which defines these characteristics were collected from asexual reproductions carried out in Enkhuizen, Netherlands. The plant history was taken on 30 week old plants, grown in window boxes in Enkhuizen, The Netherlands with day temperatures ranging from 16 to 30 ° C., and night temperatures ranging from 10 to 16 ° C. Color references are primarily to the R.H.S. Color Chart of The Royal Horticultural Society of London where general terms of ordinary dictionary significance are used.

TABLE 1
COMPARISON BETWEEN THE NEW CULTIVAR ‘SANDEAL’
AND ‘SANTANIS’

| | | | ‘Santanis’ (U.S. Plant | |
|---|---|---|---|---|
| | ‘Sandeal’ | | Patent No. 14.924) | |
| Flower size | 1.3-1.5 | cm | 1.3-1.8 | cm |
| Plant height | 23 | cm | 18 | cm |
| Plant width | 90 | cm | 70 | cm |
| Internode length | 1.5-5 | cm | 2.8-4.8 | cm |

| | | |
|---|---|---|
| Leaf color | RHS 137A | RHS 139A |

### THE PLANT

Classification —botanical: Sanvitalia x hybrida cv. ‘Sandeal’
Parentage:
   Female parent.—Seedling selection of Sanvitalia x hybrida, identified under number ‘A0178A-1,’ which is not patented.
   Male parent.—Unidentified selection of Sanvitalia x hybrida.
   Breeders code of the new plant.—‘B0240-12’.
Propagation:
   Type of cuttings.—Terminal cuttings.
   Time to initiate roots.—14 days at air temperature of 21 ° C.
   Time to develop roots.—21 days at air temperature of 17 ° C.
Plant description:
   Growth habit.—Very compact growing, bit trailing, less creeping than ‘Santanis’.
   Plant.—height 23 cm.
   Vigor.—Medium, slightly more than ‘Santanis’.
   Spreading area of plant.—90 cm.
   Strength.—Good tolerance to heat, cold, diseases and pests.
   Branching character.—Feely branching without pinching.
   Number of branches per plant.—30 branches/plant.
   Crop time.—14 weeks to flowering from good developed rooted Cutting.
The stem:
   Diameter.—3 mm.
   Shape.—Cylindrical.
   Anthocyan pigmentation.—Present in older stems, RHS 199B.
   Length of internode.—1.5-5 cm depending on light conditions.
   Pubescence.—Some, length up to 0.3 mm.
   Color.—Young stem RHS 146D — Older stem RHS N199B.
The foliage:
   Arrangement.—Opposite.
   Shape of leaf.—Lanceolate.
   Leaf apex.—Rounded.
   Leaf base.—Rounded.
   Texture.—Sturdy.
   Leaf length.—±1.8 cm.
   Leaf width.—0.7-0.8 cm.
   Depth of incision.—No incision.
   Color.—Upper side RHS 137A — Lower side RHS 138B.
   Pubescence.—Little hairy.
   Petiole.—Length No.
   Venation.—Shape Pinnate — Color: RHS 138B.
   Leaf anthocyan coloration Some coloration, RHS N199B.
The flower bud:
   Peduncle length.—1-3 cm.
   Pubescence on peduncle.—many pubescent soft hairs, 2 mm long.
   Size bud.—Diameter 0.5-0.6 cm — Length 0.4 cm.
   Shape bud.—Ball shape.
   Color bud.—RHS 144B.
Inflorescence:
   Diameter.—1.3-1.5 cm.
   Form.—Capitulum with 1 series of ray florets and more series of disc florets.
   Overlapping of ray florets.—No overlapping.
   Receptacle.—Shape Convex — Height 1.5 mm — Color: RHS 144A.
   Involucre.—5 bracts in 1 series — Color : RHS 144A.
   Fragrance.—Absent.
   Number of flowers per plant.—700 flowers/plant.
   Lastingness of flowers.—±25 days.
Ray florets:
   No. of ray florets per inflor.—13, occasionally 12 or 14.
   Size.—Length: 4-6 mm — Width: 1.5-2.5 mm.
   Shape of florets.—Ovate.
   Color.—RHS9A.
Disc florets:
   No. of disc florets per inflor.—>100.
   Size.—Length 2 mm — Width : 1mm.
   Shape of florets.—tubular.
   Color.—RHS 12A
Androecium:
   Anther color.—RHS 153B.
   Pollen color.—RHS 7A.
   Amount of pollen.—Few pollen.
Gynoecium:
   Pistil number.—1 in each floret.
   Style color.—RHS 12A.
   Stigma color.—RHS 12A.
Seed:
   Production.—10 -13 seeds per capitulum.
   Size.—Length: 2 mm — Width : 1 mm.
   Color : RHS 200A.
Disease resistance and tolerance for weather conditions: Good tolerance to heat. Strong resistance to pests and diseases.

1. A new and distinct variety of Sanvitalia plant as substantially illustrated and described herein.
